(19) United States
(12) Patent Application Publication
(10) Pub. No.: US 2026/0191923 A1
(43) Pub. Date: Jul. 9, 2026

(54) COMPOSITIONS

(71) Applicant: GIVAUDAN SA, VERNIER (CH)

(72) Inventors: Ling MA, East Hanover, NJ (US); Laura GRIFFIN, Port St. Lucie, FL (US); Sandra NARANJO MODAD, Avignon (FR)

(21) Appl. No.: 19/130,661

(22) PCT Filed: Nov. 7, 2023
(86) PCT No.: PCT/EP2023/080992

§ 371 Date: May 16, 2025

Related U.S. Application Data
(60) Provisional application No. 63/384,009, filed on Nov. 16, 2022.

(51) Int. Cl. A61K 36/185; A61P 1/00

(52) U.S. Cl. CPC A61K 36/185; A61P 1/00

(57) ABSTRACT

The present invention relates to an acerola fruit extract. In particular, the invention relates to biological, therapeutic effects and non-therapeutic effects of an acerola fruit extract and compositions comprising the extract.

### FIELD OF THE INVENTION

The present invention relates to an acerola fruit extract. In particular, the invention relates to biological, therapeutic effects and non-therapeutic effects of an acerola fruit extract and compositions comprising the extract.

### BACKGROUND OF INVENTION

It is estimated that 10¹⁴ intestinal microorganisms, belonging to over 1000 different species, are distributed along the human gastrointestinal tract, with the largest population being present in the colon. Alterations in the intestinal microbiota composition, due to lifestyle, age, diet or immuno-logical factors may bring several health problems, like intestinal and gastric disorders, as well as susceptibility to other diseases. The consumption of products containing probiotic microorganisms, fibres, and/or many other bioactive compounds has been an option to achieve a favourable composition of the intestinal microbiota, thereby ensuring a better quality of life.

Research on human nutrition has led to an awareness of the health benefits of dietary supplements. It is recognised that dietary supplements containing complex arrays of naturally occurring bioactive compounds may confer significant long-term health benefits.

In recent years, plant-origin bio-active compounds in foods (staple crops, fruit, vegetables, and others) have been gaining interest, and processes to consider them for public health recommendations are being presented and discussed in the literature. However, at times, it may be challenging to demonstrate causality, and there often is not a single compound-single effect relationship. Furthermore, it was suggested that health benefits may be due to metabolites produced by the host or gut microbiome rather than the food constituent per se.

Probiotics are live microorganisms (such as bacteria and yeasts) that provide health benefits when you consume them. They are naturally present in some fermented foods, added to some food products, and available as dietary supplements. Dietary supplements labeled as probiotics contain a wide variety of microorganisms and amounts. Many of these supplements have not been studied, so their health effects, if any, are not known.

In addition to probiotics, there are several bioactive compounds like polyphenols and fibres, which besides having the ability to improve the probiotic viability through the gastrointestinal conditions can modulate the colon microbiota composition or activity.

However, there is still a need to identify bioactive compounds that may have advantageous effects on the gut microbiota and in the treatment or prevention of digestive diseases.

### SUMMARY OF THE INVENTION

The authors of the present invention have investigated the biological effects of an acerola fruit extract and the impact of these products on the human gut microbiome. They observed that a red acerola significantly increased propionate (a short-chain fatty acid) production compared to the sugar and ascorbic acid controls and that a red and or a green acerola extract significantly increased butyrate (another short-chain fatty acid) production compared to sugar and ascorbic acid controls. These findings are quite profound because the dosage of acerola products was lower than that of ascorbic acid, suggesting that something in the acerola matrix, or the naturally sourced vitamin C, provides benefit to the microbiome that could not be achieved with a synthetic counterpart. In addition, the authors of the present invention have discovered that the acerola products shifted abundant families in a different way compared to ascorbic acid and the sugar controls.

Thus, in a first aspect, the present invention relates to an acerola fruit extract for use in a method for promoting a propionogenic or butyrogenic effect in the gut of a subject or for increasing the production of SCFAs by the gut microbiota in a subject.

In a second aspect, the present invention relates to a non-therapeutic method for increasing gastrointestinal regularity in a subject, or for increasing the SCFA content, B vitamins and/or essential fatty acids in a subject, the method comprising administering to the subject an acerola fruit extract.

In a third aspect, the present invention relates to a red acerola fruit extract for use in a method for promoting the growth of microorganisms of the genuses Bacteroides, Anaerostipes, Lachnoclostridium, Parasutterella, Bifidobacterium, Faecalibacterium, Parabacteroides, UCG-002, Blautia, CAG-56, Alistipes and Phascolarctobacterium and the family Lachnospiraceae in the gut microbiome.

In a fourth aspect, the present invention relates to a green acerola fruit extract for use in a method for promoting the growth of microorganisms of the genuses Bacteroides, Anaerostipes, Lachnoclostridium, Parasutterella, Bifidobacterium, Faecalibacterium, Parabacteroides, Dorea, UCG-002, Blautia, and Alistipes, and the family Lachnospiraceae in the gut microbiome.

In a further aspect, the present invention relates to a method of increasing the population of genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome comprising directly delivering to the large intestine of red acerola fruit extract.

In a certain embodiment, the person is experiencing or is at risk of experiencing high cholesterol levels.

In a further aspect, the present invention relates to a method of increasing the population of the genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome comprising directly delivering to the large intestine of green acerola fruit extract.

In a certain embodiment, the person is experiencing or is at risk of experiencing at least one of the following conditions gastrointestinal infections, intestinal mucosal health problems, gut barrier integrity probelms, congenital chloride diarrhea, cholera, colorectal cancer, inflammatory bowel disease, colon heartburn, cancer, irritable bowel syndrome, histamine intolerance and lactose intolerance, gallstones, cholecystitis and cholangitis, rectal problems (anal fissure, haemorrhoids, proctitis and rectal prolapse).

In a certain embodiment, the population of the genuses and/or families was decreased prior to direct delivery of the acerola extract.

In a certain embodiment, a short chain fatty acid in the intestine is increased.

In a further aspect, the present invention relates to a cosmetic, nutraceutical or cosmeceutical composition, a food supplement or dietary supplement comprising an acerola fruit extract.

### DESCRIPTION OF THE FIGURES

FIG. 1: Effect of the administration of compositions comprising red acerola extract, green acerola extract, sugar control (55% fructose:45% glucose) and ascorbic acid on the levels of propionate (A) and butyrate (B).

FIG. 2: 2a) Heat map displaying the abundances of different bacterial families after the treatments. AA is Ascorbic acid. 2b) Heat map clustering analysis in all the 5 groups at the family level. The green/red acerola upregulated beneficial bacterial families such as Lacnospiraceaea, Clostridaceae, Barnesiellaceae, Erysipelatrichaceae and some of Bacteroidales and some members of lachnospiraceae family. Most significantly (P<0.05) higher genera are highlighted using plus symbol (+) and most significantly lower are highlighted using minus symbol (−). Controls: blank control (Blank), Ascorbic acid (AA), Fructose/glucose mix (sugar control).

FIG. 3: Beta diversity using principle coordinate analysis using Bray-Curtis dissimilarity by permutational multivariate analysis of variance (PERMANOVA) statistical method significant divergence (R2=0.9, p=0.001) among the groups by taxonomic composition between green/red acerola and controls, clearly green/red acerola triggered the microbiome shifts. P<0.05 is significant. Controls: blank control (Blank), Ascorbic acid, Fructose (Fruc)/glucose (Gluc) mix (sugar control).

FIG. 4: Linear discrimination analysis effect size (LefSe, log LDA>2.0) analysis in all the 5 groups at genus level. The values are shown as the mean±SEM with P<0.05. Controls: blank control (Blank), Ascorbic acid (absorbable fraction), Fructose/glucose mix (sugar control). The green/red acerola upregulated beneficial bacterial genera such as Faecalibacterium, Anaerostipes and some genera of Lachnoclostridium and some members of lachnospiraceae family.

### DESCRIPTION OF THE INVENTION

The authors of the invention have discovered that an acerola fruit extract has different biological effects which are associated with therapeutic effects. In addition, the authors have discovered that the acerola fruit extract has an impact on the human gut microbiome and it can promote the growth of different microorganisms.

In a first aspect, the present invention relates to an acerola fruit extract for use in a method (hereinafter “the first method of the invention”) for promoting a propionogenic or butyrogenic effect in the gut of a subject or for increasing the production of SCFAs by the gut microbiota in a subject.

Malpighia emarginata is a tropical fruit-bearing shrub or small tree in the family Malpighiaceae. Common names include acerola cherry, Guarani cherry, Barbados cherry, West Indian cherry, and wild crepe myrtle. Acerola is native to Paraguay and Brazil in South America, Central America and southern Mexico, Puerto Rico, Dominican Republic and Haiti. It is a rich source of vitamin C. Acerola also contains many minerals and other vitamins, including beta-carotene, lutein, thiamine, riboflavin, niacin, pyridoxine, folic acid, and pantothenic acid. Acerola is commonly used to prevent vitamin C deficiency.

The term “acerola fruit extract” refers to the extract obtained from the acerola fruit, including the whole fruit, and/or skin of the acerola fruit, and/or the pulp and/or the residues of the fruit after juicing of the fruit. The fruit or the parts may be fresh, frozen or dried. Juice that is recovered from any processing of the fruit any also be processed to recover an acerola fruit extract.

In a particular embodiment, the acerola fruit extract is an acerola fruit powder. In a more particular embodiment, the acerola fruit extract is an acerola juice powder.

In another particular embodiment, the acerola fruit extract contains vitamin C, antioxidant polyphenolic compounds, fiber, glucose, fructose, anthocyanin and polyunsaturated fatty acids (PUFA).

Vitamin C (also known as ascorbic acid and ascorbate) is a water-soluble vitamin found in citrus and other fruits and vegetables, and also sold as a dietary supplement. Vitamin C is an essential nutrient involved in the repair of tissue, the formation of collagen, and the enzymatic production of certain neurotransmitters. It is required for the functioning of several enzymes and is important for immune system function. It also functions as an antioxidant.

Polyphenols are compounds with aromatic ring and several hydroxyl groups and often have functional groups beyond hydroxyl groups. Polyphenolic compounds are usually referred to as a diverse group of naturally occurring compounds containing multiple phenolic functionalities. In a particular embodiment, the polyphenolic compounds are selected from the group consisting of: p-Coumaric acid, Ferulic acid, Rutin, Caffeic acid, Catechin, Epicatechin, Gallocatechin gallate, Quercetin,

Polyunsaturated fatty acids (PUFA) are fatty acids that contain more than one double bond in their backbone.

In a particular embodiment, the PUFA is polyunsaturated fatty acids (PUFA), with 18 carbon atoms. In a more particular embodiment, the PUFA is selected from the group consisting of: alpha-linolenic acid (ALA), stearidonic acid (SDA), linoleic acid (LA), gamma-linolenic acid (GLA), oleic acid, a conjugated linoleic acid or a conjugated linolenic acid. In a more particular embodiment the conjugated linoleic acid is rumenic acid and the conjugated linolenic acid is selected of the group consisting of: α-calendic acid, β-Calendic acid, jacaric acid, α-eleostearic acid, β-eleostearic acid, catalpic acid, punicic acid, α-parinaric or β-parinaric acid.

In another particular embodiment, the PUFA is a long-chain polyunsaturated fatty acids (LC-PUFA) with 20 or more carbon atoms. In a more particular embodiment, the LC-PUFA is selected from the group consisting of: eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, cervonic acid), tetracosapentaenoic acid, eicosadienoic acid, arachidonic acid (AA) or adrenic acid (AdA). In another particular embodiment, the acerola fruit extract contains at least 20% vitamin C. In a more particular embodiment, the acerola fruit extract contains between 20% and 60% of vitamin C.

In another particular embodiment, the acerola fruit extract contains at least 4,5% of antioxidant polyphenolic compounds. In a more particular embodiment, the acerola fruit extract contains between 4,5% and 30% of polyphenolic compounds. In a more particular embodiment, the acerola fruit extract contains between 100 and 300 mg of gallic acid equivalents per gram of extract of polyphenolic compounds

In yet another embodiment, the acerola fruit extract is a red acerola fruit extract that contains between 10 and 300, between 20 and 290, between 30 and 280, between 40 and 270, between 50 and 260, between 60 and 250, between 70 and 240, between 80 and 230, between 90 and 220, between 100 and 210, between 110 and 200, between 110 and 190, between 110 and 180, between 110 and 170, between 110 and 160, between 110 and 150, between 110 and 140, between 110 and 130, between 110 and 120 mg of gallic acid equivalents per gram of extract.

In yet another embodiment, the acerola fruit extract is a green acerola fruit extract that contains between 10 and 500, between 20 and 490, between 30 and 480, between 40 and 470, between 50 and 460, between 60 and 450, between 70 and 440, between 80 and 430, between 90 and 420, between 100 and 410, between 110 and 400, between 120 and 390, between 130 and 380, between 140 and 370, between 150 and 360, between 160 and 350, between 170 and 340, between 180 and 330, between 190 and 320, between 200 and 310, between 210 and 300, between 220 and 290, between 230 and 280, between 240 and 270, between 240 and 260, between 240 and 250 mg of gallic acid equivalents per gram of extract.

In another particular embodiment, the acerola fruit extract contains at least 0.5% of total fiber. In a more particular embodiment, the acerola fruit extract contains between 0.5% and 10% of total fiber.

In another particular embodiment, the acerola fruit extract contains at least 5% of the combined content of glucose and fructose. In a more particular embodiment, the acerola fruit extract contains between 5% and 50% of the combined content of glucose and fructose.

In another particular embodiment, the acerola fruit extract contains at least 1000 ppm of PUFA. In a more particular embodiment, the acerola fruit extract contains between 1000 ppm and 4000 ppm of PUFA.

In another particular embodiment, the acerola fruit extract may contain other components such as a pH modifier or an acidity regulator. pH modifiers, acidity regulators, or pH control agents, are additives used to change or maintain pH (acidity or basicity). They can be organic or mineral acids, bases, neutralizing agents, or buffering agents. In a particular embodiment, the acerola fruit extract contains magnesium hydroxide as an acidity regulator.

The acerola fruit extract can be green or red acerola fruit extract. The colour difference between red and green acerola, is attributed to the ripeness of the fruit. Green acerola is harvested whilst the fruit is still unripe, and red acerola is harvested once the fruit has fully matured.

Thus, in a particular embodiment, the acerola fruit extract for use in the first method of the invention is a red acerola fruit extract.

In another particular embodiment, the acerola fruit extract for use in the first method of the invention is a green acerola fruit extract.

The quantity of vitamin C, antioxidant polyphenolic compounds, fiber, glucose, fructose and polyunsaturated fatty acids (PUFA) may differ between the red acerola fruit extract and the green acerola fruit extract.

Thus, in a particular embodiment, the green acerola fruit extract contains between 30% and 60% of vitamin C.

In another particular embodiment, the green acerola fruit extract contains between 6% and 30% of polyphenolic compounds. In yet another embodiment, the acerola fruit extract is a green acerola fruit extract that contains between 240 and 250 mg of gallic acid equivalents per gram of extract.

In another particular embodiment, the green acerola fruit extract contains between 0.5% and 4% of total fiber.

In another particular embodiment, the green acerola fruit extract contains between 5% and 15% of the combined content of glucose and fructose.

In another particular embodiment, the red acerola fruit extract contains between 20% and 40% of vitamin C.

In another particular embodiment, the red acerola fruit extract contains between 4,5% and 15% of polyphenolic compounds. In yet another embodiment, the acerola fruit extract is a red acerola fruit extract that contains between 110 and 120 mg of gallic acid equivalents per gram of extract.

In another particular embodiment, the red acerola fruit extract contains between 2% and 9% of total fiber.

In another particular embodiment, the red acerola fruit extract contains between 25% and 50% of the combined content of glucose and fructose.

In another particular embodiment, the red acerola fruit extract contains between 1700 and 3500 ppm of PUFA. In a more particular embodiment, the red acerola fruit extract comprises a combination of polyunsaturated fatty acids (PUFA). In a preferred embodiment, the red acerola fruit extract comprises a combination of oleic acid (18:1), linoleic acid (18:2) and linolenic acid (18:3).

The authors of the present invention have discovered that the red acerola significantly increased propionate production, compared to the sugar and ascorbic acid controls. Both, red and green acerola fruit extract, significantly increased butyrate production compared to sugar and ascorbic acid controls, but the strongest butyrogenic effects were attributed to green acerola.

Thus, in a particular embodiment, the acerola fruit extract for use in the first method of the invention is red acerola fruit extract and it has a propionogenic effect.

The term “propionogenic effect” refers to the stimulation of propionate production. Propionate is a short chain fatty acid (SCFA), which has the formula C₃H₅O₂. This SCFA is found in circulation in higher concentration and is associated with some health benefits such as reducing the serum cholesterol levels, inhibiting fatty acid synthesis or increasing satiety.

In another particular embodiment, the acerola fruit extract for use in the first method of the invention is a green acerola fruit extract and it has a butyrogenic effect.

The term “butyrogenic effect” refers to the stimulation of butyrate production. Butyrate is a SCFA, which has the chemical formula C₄H₇O₂. This SCFA is a primary energy source for colonocytes and supports intestinal mucosal health. Butyrate may also have anticarcinogenic properties, preventing or inhibiting colorectal cancer. Butyrate may also exert anti-inflammatory properties.

As mentioned above, the authors of the present invention have found that the administration of acerola fruit extract results in an increase in the production of SFCA. Short chain fatty acids (SCFAs) are fatty acids with fewer than six carbon atoms. In a particular embodiment, the first method of the invention increases the production of at least one SCFA selected from the group: formate, acetate, butyrate, propionate, lactate, isobutyrate, valerate, isovalerate and 2-methylbutanoate. In a more particular embodiment, the first method of the invention increases the production of at least one SCFA selected from the group: acetate, butyrate and propionate. In a preferred embodiment, the first method of the invention increases the production of butyrate and propionate.

As an expert in the field may know, many different instrumental techniques can be used to determine the SCFAs concentration; these techniques include gas chromatography (GC), gas chromatography-mass spectrometer (GC-MS), liquid chromatograph (LC), capillary electrophoresis (CE), capillary gas chromatography, ion exchange high-performance liquid chromatography (IC) and nuclear magnetic resonance (NMR). In a particular embodiment of the present invention, the SCFAs concentration is determined using capillary gas chromatography, coupled with a flame ionization detector (FID).

Short chain fatty acids (SCFAs) are commonly produced by healthy gut microbiota and they have a protective role against enteric pathogens. The term “microbiota” refers to the set of living micro-organisms found in the intestine or digestive tract of an organism. Microbiota include bacteria, archaea, protists, fungi, and viruses and have been found to be crucial for immunologic, hormonal, and metabolic homeostasis of their host. The dominant bacterial genera in the human gut are Bacteroides, Anaerostipes, Lachnoclostridium, Parasutterella, Bifidobacterium, Faecalibacterium, Parabacteroides, UCG-002, Blautia, CAG-56, Alistipes, Phascolarctobacterium, Parasutterella and Dorea.

In the present invention, the term “subject” refers to any animal of any species. Preferably the subject is a mammal. More preferably the subject is a human.

As mentioned above, the use of the red acerola fruit extract in the first method of the invention results in a propionogenic effect, which is associated with potential health benefits. Thus, in a particular embodiment, the use of the red acerola fruit extract in the first method of the invention results in a reduction of the cholesterol levels, a reduction of the fatty acid levels or an increase in satiety in a subject.

Thus, in a particular embodiment, the use of the red acerola fruit extract in the first method of the invention results in a reduction of the cholesterol levels. Cholesterol is a sterol (or modified steroid), a type of lipid. Cholesterol is biosynthesized by all animal cells and is an essential structural component of animal cell membranes. Cholesterol also serves as a precursor for the biosynthesis of steroid hormones, bile acid and vitamin D. Cholesterol is the principal sterol synthesized by all animals.

Cholesterol circulating in the blood is carried by lipoproteins. The two major cholesterol-carrying lipoproteins are low-density lipoprotein (LDL) and high-density lipoprotein (HDL):
LDL cholesterol (LDL-C) is often referred to as “bad” cholesterol because too much of it can build up in the arteries and form plaques, which increases the risk of heart disease.
HDL cholesterol (HDL-C) is often referred to as “good” cholesterol as it carries cholesterol to the liver to be broken down and excreted.

The recommended ranges for cholesterol will vary based on age and gender. As people get older, cholesterol levels rise naturally. For most healthy adults (19 and older), total cholesterol should be less than 200 mg/dL, LDL less than 100 mg/dL, and HDL greater than 40 mg/dL. For children (19 and younger), total cholesterol should be less than 170 mg/dL, LDL less than 110 mg/dL, and HDL greater than 45 mg/dL.

In a particular embodiment, the use of the red acerola fruit extract in the first method of the invention reduces the total cholesterol level and/or the LDL cholesterol level.

In another particular embodiment, the use of the red acerola fruit extract in the first method of the invention results in a reduction of the fatty acid levels. Fatty acids are long-chain carboxylic acids that may contain one or more carbon-carbon double bonds. High plasma free fatty acid levels are associated with some diseases, such as cardiovascular and heart disease, cancer, inflammation and autoimmune diseases.

Fatty acids can be saturated or unsaturated. Saturated fatty acids have no C═C double bonds. They have the same formula CH₃(CH₂)ₙCOOH, wherein n is from 4 to 28. The most important saturated fatty acids are: caprylic acid, capric acid lauric acid, myristic acid, palmitic acid, stearic acid, arachidic acid, behenic acid, lignoceric acid and cerotic acid. In a particular embodiment, the use of the red acerola fruit extract in the first method of the invention results in a reduction of saturated fatty acids levels.

Unsaturated fatty acids have one or more C═C double bonds. The C═C double bonds can give either cis or trans isomers. The most important unsaturated fatty acids are: myristoleic acid, palmitoleic acid, sapienic acid, oleic acid, elaidic acid, vaccenic acid, linoleic acid, linoelaidic acid, α-Linolenic acid, arachidonic acid, eicosapentaenoic acid and erucic acid.

In another particular embodiment, the use of the red acerola fruit extract in the first method of the invention results in an increase in satiety in a subject. The term “satiety” as used herein, refers to the psycho-biological process that suppresses hunger after an eating occasion and prevents further eating.

The acerola fruit extract must be present in an effective amount to exert its propionogenic effect. The term “effective amount” means any amount of the acerola fruit extract which, when administered to a subject, is sufficient to produce the desired effect. The effective amount may vary depending on, for example, the age, body weight, general health, sex and diet of the subject, as well as the mode and timing of administration. For example, an effective amount may be from about 1 g to 10 g of green acerola extract, such as from about 4 to 8 g, such as 6 g of acerola extract.

As mentioned above, the authors of the present invention have discovered that the green acerola significantly increased butyrate production, i.e. they discovered that the use of a green acerola fruit extract has a butyrogenic effect, which is associated with potential health benefits, in particular in digestive diseases.

The term “digestive diseases” refers to various disorders of the digestive system. These conditions can range from mild to severe. In certain embodiments, the disorder of the digestive system is selected from one or more of heartburn, cancer, irritable bowel syndrome, histamine intolerance and lactose intolerance. Other digestive diseases include: gallstones, cholecystitis and cholangitis, rectal problems (anal fissure, haemorrhoids, proctitis and rectal prolapse), oesophageal problems (strictures, achalasia and oesophagitis), stomach problems (gastritis, gastric ulcers usually caused by Helycobacter Pylori infection and cancer), liver problems (hepatitis B, hepatitis C, cirrhosis, liver failure and alcoholic and autoimmune hepatitis), pancreatitis and pancreatic pseudocyst, intestinal problems such as polyps and cancer, infections, celiac disease, Crohn's disease, ulcerative colitis, diverticulosis, malabsorption, short bowel syndrome and intestinal ischemia, gastroesophageal reflux disease (GERD), peptic ulcer disease and hiatal hernia, among others.

In a particular embodiment, the use of a green acerola fruit extract in the first method of the invention is associated with health benefits such as restoring intestinal mucosal health, increases gut barrier integrity, results in the symptomatic treatment of congenital chloride diarrhea, results in the symptomatic treatment of cholera, results in the treatment or prevention of colorectal cancer or results in the prevention or treatment of an inflammatory bowel disease of the colon in a subject.

Thus, in a particular embodiment, the use of a green acerola fruit extract in the first method of the invention may help restore intestinal mucosal health. The term “intestinal mucosal health” refers to the property of the intestinal mucosa that ensures adequate containment of undesirable luminal contents within the intestine while preserving the ability to absorb nutrients. Its role in protecting mucosal tissues and the circulatory system from exposure to pro-inflammatory molecules, such as microorganisms, toxins, and antigens is vital for the maintenance of health and well-being. Alteration of the mucosal barrier function with accompanying increased permeability and/or bacterial translocation has been linked with a variety of conditions, such as microbial infections, irritable bowel syndrome, inflammatory bowel disease, celiac disease, metabolic syndrome, non-alcoholic fatty liver disease, diabetes, and septic shock.

The intestinal mucosal health can be altered by dietary alterations, medical treatments, microbial infections or chronic illnesses. In a particular embodiment, the intestinal mucosal health is altered due to gastroenteritis. Gastroenteritis is inflammation of the stomach, small intestine, or large intestine, leading to a combination of abdominal pain, cramping, nausea, vomiting, and diarrhea. Acute gastroenteritis usually lasts fewer than 14 days. This is in contrast to persistent gastroenteritis, which lasts between 14 and 30 days, and chronic gastroenteritis, which lasts more than 30 days.

In a more particular embodiment, the intestinal mucosal health is altered due to acute gastroenteritis. Acute gastroenteritis may be caused by many infectious agents. In a particular embodiment the acute gastroenteritis may be caused by a virus, a bacteria or a parasite. In a more particular embodiment the acute gastroenteritis may be caused by a virus from the genus Norovirus, Rotavirus, Adenovirus or Astrovirus, by a bacteria from the genus Escherichia, Bacillus, Shigella, Salmonella or Yersinia, or by a parasite from the genus Giarida, Cryptosporidium, Amebiasis or Entamoeba.

In another particular embodiment, the use of a green acerola fruit extract in the first method of the invention may increases gut barrier integrity. Pathophysiological or environmental factors may be the crucial factors that usurp normal physiology and affects to the gut barrier integrity. Some of these factors are environmental stress, infections caused by pathogens, the consumption of drugs or antibiotics or alterations in the diet.

Intestinal permeability is a measure of intestinal mucosal barrier function and is defined as “the ease with which the intestinal epithelium allows molecules to pass through by unmediated passive diffusion”. There are several non-invasive methods to measure intestinal permeability. One is to measure the passage of dietary proteins through the intestine into the blood. If these proteins are elevated in the blood, intestinal permeability is increased. Two proteins are commonly used: egg ovalbumin and milk beta-lactoglobulin. Generally, when these proteins enter the blood, the body produces antibodies against them. Thus, the degree of intestinal mucosal integrity can be measured by studying intestinal permeability, which can be determined by determining the presence of beta-lactoglobulin protein or antibodies generated against it in the blood. An enzyme-immunoassay technique, also known as ELISA, can be used.

Another method is the measure of lipopolysaccharides. The presence of specific antibodies in serum can also be a sign of intestinal permeability. Lipopolysaccharide (LPS) is a naturally occurring endotoxin found in the gut, genitourinal, and respiratory tracts. As part of the cell wall of Gram-negative organisms, it is expressed when the organism's cell membrane is shed or ruptured. A healthy mucosal layer with intact tight junctions prevents the paracellular translocation of LPS. The presence of LPS and LPS IgA, IgG, and IgM antibodies in the blood is clinically relevant when attempting to identify the degree of intestinal barrier permeability.

In another particular embodiment, the use of a green acerola fruit extract according to the first method of the invention may results in the symptomatic treatment of congenital chloride diarrhea. Congenital chloride diarrhea (CCD) is a lifelong condition that causes large, watery stools (diarrhea) that contain an excess of chloride. CCD causes electrolyte imbalances including low blood sodium levels (hyponatremia) and chloride levels (hypochloremia). Electrolyte imbalances and severe dehydration can lead to volume depletion (too little fluid surrounding body cells), loss of acid from the blood (metabolic alkalosis), hyperaldosteronism, delayed growth and development, and kidney damage (nephropathy).

The expression “symptomatic treatment of congenital chloride diarrhea” refers to the reduction of the symptoms associated with the disease, i.e. the use of the green acerola fruit extract in the first method of the invention may reduce the symptoms associated with the CCD, but do not seem to have an effect in the underlying infection.

In another particular embodiment, the use of the green acerola fruit extract in the first method of the invention may help the symptomatic treatment of cholera. Cholera is an acute diarrheal infection caused by ingestion of food or water contaminated with the bacterium Vibrio cholerae. Cholera is an extremely virulent disease that can cause severe acute watery diarrhea. There are many serogroups of V. cholerae, but only two—O1 and O139—cause outbreaks.

The expression “symptomatic treatment of cholera” refers to the reduction of the symptoms associated with the disease, i.e. the use of the green acerola fruit extract in the first method of the invention may reduce the symptoms associated with cholera, but do not seem to have an effect in the underlying infection.

In another particular embodiment, the use of the green acerola fruit extract in the first method of the invention may contribute to the treatment or prevention of colorectal cancer.

The term “treatment” as used in the present invention refers to the treatment of a disease or medical condition in a subject, preferably human, comprising:
(a) preventing the onset of the disease or medical condition, i.e. prophylactic treatment of a subject;
(b) ameliorating the disease or medical condition, i.e. causing regression of the disease or medical condition in a subject;
(c) to suppress the disease or medical condition, i.e. to slow down the development of the disease or medical condition in a subject; or
(d) to alleviate the symptoms of the disease or medical condition in a subject.

The term “prevention” refers to preventing the occurrence of the disease, i.e. preventing the occurrence of the disease or pathological condition in a subject (preferably a mammal, and more preferably a human), in particular, where such a subject is predisposed to the pathological condition.

The term “cancer” refers to a set of related diseases in which some of the body's cells begin to divide without stopping and spread to surrounding tissues, such as breast cancer, colorectal cancer and lung cancer. This division may start locally and spread to other surrounding tissues. Colorectal cancer, also known as bowel cancer, colon cancer, or rectal cancer, is the development of cancer from the colon or rectum (parts of the large intestine). In a preferred embodiment, the colorectal cancer is an adenocarcinoma, which is a cancer of the cells that line the inside surface of the colon, or a carcinoid tumour, which start in hormone-producing cells in the intestines In another preferred embodiment, the use of the green acerola fruit extract in the first method of the invention may help the treatment or prevention of a stage I cancer, wherein the cancer has not grown deeply into nearby tissues.

In another preferred embodiment, the use of the green acerola fruit extract in the first method of the invention potentially assist in the treatment or prevention of a stage II or a stage III cancer, wherein the cancer has grown more deeply into nearby tissue. They may have also spread to lymph nodes but not to other parts of the body.

In another particular embodiment, the use of the green acerola fruit extract in the first method of the invention may help the prevention or treatment of an inflammatory bowel disease of the colon in a subject. Inflammatory bowel disease is a term that describes disorders involving long-standing (chronic) inflammation of tissues in the digestive tract. Types of IBD include ulcerative colitis and Crohn's disease. Thus, in a particular embodiment, the inflammatory bowel disease is ulcerative colitis or Crohn's disease. Ulcerative colitis involves inflammation and sores (ulcers) along the lining of your large intestine (colon) and rectum. Crohn's disease is characterized by inflammation of the lining of your digestive tract, which often can involve the deeper layers of the digestive tract. Crohn's disease most commonly affects the small intestine. However, it can also affect the large intestine and uncommonly, the upper gastrointestinal tract.

The acerola fruit extract must be present in an effective amount to exert its butyrogenic effect. The term “effective amount” means any amount of the acerola fruit extract which, when administered to a subject, is sufficient to produce the desired effect. The effective amount may vary depending on, for example, the age, body weight, general health, sex and diet of the subject, as well as the mode and timing of administration. For example, an effective amount may be from about 0.5 g to 10 g of green acerola extract, such as from about 4 to 8 g, such as 6 g of acerola extract.

In another aspect, the present invention relates to a non-therapeutic method (hereinafter “the non-therapeutic method of the invention”) for increasing gastrointestinal regularity in a subject, or for increasing the SCFA content, B vitamins and/or essential fatty acids in a subject, the method comprising administering to the subject an acerola fruit extract.

The terms “acerola”, “acerola fruit extract” and “subject” have been defined or explained above, and these definitions are applicable to the non-therapeutic method. In a particular embodiment, the acerola fruit extract is an acerola fruit powder. In a more particular embodiment, the acerola fruit extract is an acerola juice powder.

In a particular embodiment, the acerola fruit extract contains vitamin C, antioxidant polyphenolic compounds, fiber, glucose, fructose and polyunsaturated fatty acids (PUFA).

The terms “vitamin C”, “polyphenolic compounds” and “PUFA” have been defined or explained above, and these definitions are applicable to the non-therapeutic method of the invention.

In another particular embodiment, the acerola fruit extract contains at least 20% vitamin C. In a more particular embodiment, the acerola fruit extract contains between 20% and 60% of vitamin C.

In another particular embodiment, the acerola fruit extract contains at least 4,5% of antioxidant polyphenolic compounds. In a more particular embodiment, the acerola fruit extract contains between 4,5% and 30% of polyphenolic compounds.

In yet another embodiment, the acerola fruit extract is a red acerola fruit extract that contains between 10 and 300, between 20 and 290, between 30 and 280, between 40 and 270, between 50 and 260, between 60 and 250, between 70 and 240, between 80 and 230, between 90 and 220, between 100 and 210, between 110 and 200, between 110 and 190, between 110 and 180, between 110 and 170, between 110 and 160, between 110 and 150, between 110 and 140, between 110 and 130, between 110 and 120 mg of gallic acid equivalents per gram of extract.

In yet another embodiment, the acerola fruit extract is a green acerola fruit extract that contains between 10 and 500, between 20 and 490, between 30 and 480, between 40 and 470, between 50 and 460, between 60 and 450, between 70 and 440, between 80 and 430, between 90 and 420, between 100 and 410, between 110 and 400, between 120 and 390, between 130 and 380, between 140 and 370, between 150 and 360, between 160 and 350, between 170 and 340, between 180 and 330, between 190 and 320, between 200 and 310, between 210 and 300, between 220 and 290, between 230 and 280, between 240 and 270, between 240 and 260, between 240 and 250 mg of gallic acid equivalents per gram of extract.

In another particular embodiment, the acerola fruit extract contains at least 0.5% of total fiber. In a more particular embodiment, the acerola fruit extract contains between 0.5% and 10% of total fiber.

In another particular embodiment, the acerola fruit extract contains at least 5% of the combined content of glucose and fructose. In a more particular embodiment, the acerola fruit extract contains between 5% and 50% of the combined content of glucose and fructose.

In another particular embodiment, the acerola fruit extract contains at least 1000 ppm of PUFA. In a more particular embodiment, the acerola fruit extract contains between 1000 ppm and 4000 ppm of PUFA.

In a particular embodiment, the PUFA is a polyunsaturated fatty acid (PUFA), with 18 carbon atoms. In a more particular embodiment, the PUFA is selected from the group consisting of: alpha-linolenic acid (ALA), stearidonic acid (SDA), linoleic acid (LA), gamma-linolenic acid (GLA), oleic acid, a conjugated linoleic acid or a conjugated linolenic acid. In a more particular embodiment the conjugated linoleic acid is rumenic acid and the conjugated linolenic acid is selected of the group consisting of: α-calendic acid, β-Calendic acid, jacaric acid, α-eleostearic acid, β-eleostearic acid, catalpic acid, punicic acid, α-parinaric or β-parinaric acid.

In another particular embodiment, the PUFA is a long-chain polyunsaturated fatty acids (LC-PUFA) with 20 or more carbon atoms. In a more particular embodiment, the LC-PUFA is selected from the group consisting of: eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, cervonic acid), tetracosapentaenoic acid, eicosadienoic acid, arachidonic acid (AA) or adrenic acid (AdA).

In another particular embodiment, the acerola fruit extract may contain other components such as a pH regulator or a pH modifier, or an acidity regulator. The term “pH regulator” or “acidity regulator” has been defined or explained above, and this definition is applicable to the non-therapeutic method of the invention. In a particular embodiment, the acerola fruit extract contains magnesium hydroxide as am acidity regulator.

The acerola fruit extract can be green or red acerola fruit extract. Thus, in a particular embodiment, the acerola fruit is red acerola fruit extract or green acerola fruit extract.

The quantity of vitamin C, antioxidant polyphenolic compounds, fiber, glucose, fructose and polyunsaturated fatty acids (PUFA) may differ between the red acerola fruit extract and green acerola fruit extract.

Thus, in a particular embodiment, the green acerola fruit extract contains between 30% and 60% of vitamin C.

In another particular embodiment, the green acerola fruit extract contains between 6% and 30% of polyphenolic compounds. In yet another embodiment, the green acerola fruit extract contains between 240 and 250 mg of gallic acid equivalents per gram of extract.

In another particular embodiment, the green acerola fruit extract contains between 0.5% and 4% of total fiber.

In another particular embodiment, the green acerola fruit extract contains between 5% and 15% of the combined content of glucose and fructose.

As mentioned above, the quantity of vitamin C, antioxidant polyphenolic compounds, fiber, glucose, fructose and polyunsaturated fatty acids (PUFA) may differ between the red acerola fruit extract and green acerola fruit extract.

Thus, in a particular embodiment, the red acerola fruit extract contains between 20% and 40% of vitamin C.

In another particular embodiment, the red acerola fruit extract contains between 4,5% and 15% of polyphenolic compound. In yet another embodiment, the red acerola fruit extract contains between 110 and 120 mg of gallic acid equivalents per gram of extract.

In another particular embodiment, the red acerola fruit extract contains between 2% and 9% of total fiber,

In another particular embodiment, the red acerola fruit extract contains between 25% and 50% of the combined content of glucose and fructose

In another particular embodiment, the red acerola fruit extract contains between 1700 and 3500 ppm of PUFA. In a more particular embodiment, the red acerola fruit extract comprises a combination of polyunsaturated fatty acids (PUFA). In a preferred embodiment, the red acerola fruit extract comprises a combination of oleic acid (18:1), linoleic acid (18:2) and linolenic acid (18:3).

As mentioned above, in a particular embodiment, the non-therapeutic method of the invention increases gastrointestinal regularity in a subject.

In another particular embodiment, the non-therapeutic method of the invention increases the level of SCFAs in a subject. In a preferred embodiment, the non-therapeutic method of the invention increases the production of at least one SCFA selected from the group: formate, acetate, butyrate, propionate, lactate, isobutyrate, valerate, isovalerate and 2-methylbutanoate. In a more preferred embodiment, the non-therapeutic method of the invention increases the production of at least one SCFA selected from the group: acetate, butyrate and propionate. In an even more preferred embodiment, the non-therapeutic method of the invention increases the production of butyrate and propionate.

In another particular embodiment, the non-therapeutic method increases the level of B vitamins in a subject. B vitamins are a class of water-soluble vitamins that play important roles in cell metabolism and synthesis of red blood cells. Each B vitamin is either a cofactor (generally a coenzyme) for key metabolic processes or is a precursor needed to make one and is thus an essential nutrient. In a preferred embodiment, the non-therapeutic method increases the level of at least one of the B vitamins selected from the group: vitamin B1 (thiamine), vitamin B2 (riboflavin), vitamin B3 (niacin), vitamin B5 (pantothenic acid), vitamin B6 (pyridoxine, pyridoxal), vitamin B7 (biotin), vitamin B9 (folate) and vitamin B12 (cobalamin).

In another particular embodiment, the non-therapeutic method increases the level of essential fatty acids in a subject. Essential fatty acids, or EFAs, are fatty acids that humans and other animals must ingest because the body requires them for good health but cannot synthesize them. The term “essential fatty acid” refers to fatty acids required for biological processes but does not include the fats that only act as fuel. Only two fatty acids are known to be essential for humans: alpha-linolenic acid (an omega-3 fatty acid) and linoleic acid (an omega-6 fatty acid). Some other fatty acids are sometimes classified as “conditionally essential”, meaning that they can become essential under some developmental or disease conditions; examples include docosahexaenoic acid (an omega-3 fatty acid) and gamma-linolenic acid (an omega-6 fatty acid). In a more particular embodiment, the non-therapeutic method increases the level of at least one of the essential fatty acids selected from the group: alpha-linolenic acid (an omega-3 fatty acid), linoleic acid (an omega-6 fatty acid), docosahexaenoic acid (an omega-3 fatty acid) and gamma-linolenic acid (an omega-6 fatty acid).

The acerola fruit extract must be present in an effective amount to exert its non-therapeutic effect. The term “effective amount” means any amount of the acerola fruit extract which, when administered to a subject, is sufficient to produce the desired effect. The effective amount may vary depending on, for example, the age, body weight, general health, sex and diet of the subject, as well as the mode and timing of administration. For example, an effective amount may be from about 0.5 g to 10 g of red acerola extract, such as from about 4 to 8 g, such as 6 g of acerola extract.

The authors of the present invention have discovered that the acerola fruit extract has an impact on the gut microbiome. They have discovered that the use of an acerola fruit extract promotes the growth of microorganisms in the gut microbiome.

The gut microbiome refers to the totality of microorganisms, bacteria, viruses, protozoa, and fungi, and their collective genetic material present in the gastrointestinal tract (GIT). Human gut microbiome is unprecedentedly complex and diverse with the majority of bacteria from the four phyla Bacteroides, Firmicutes, Proteobacteria, and Actinobacteria, constituting more than 98% of the microbes. Most bacteria belong to the genera Bacteroides, Clostridium, Faecalibacterium, Eubacterium, Ruminococcus, Peptococcus, Peptostreptococcus, and Bifidobacterium. Other genera, such as Escherichia and Lactobacillus, are present to a lesser extent. Species from the genus Bacteroides alone constitute about 30% of all bacteria in the gut, suggesting that this genus is especially important in the functioning of the host. Fungal genera that have been detected in the gut include Candida, Saccharomyces, Aspergillus, Penicillium, Rhodotorula, Trametes, Pleospora, Sclerotinia, Bullera, and Galactomyces, among others.

The authors of the present invention have discovered that the use of a red or a green acerola fruit extract results in a change of the microbiota composition.

Thus, in another aspect, the present invention relates to a red acerola fruit extract for use in a method (hereinafter the second method of the invention) for promoting the growth of microorganisms of the genuses Bacteroides, Anaerostipes, Lachnoclostridium, Parasutterella, Bifidobacterium, Faecalibacterium, Parabacteroides, UCG-002, Blautia, CAG-56, Alistipes and Phascolarctobacterium or the family Lachnospiraceae in the gut microbiome.

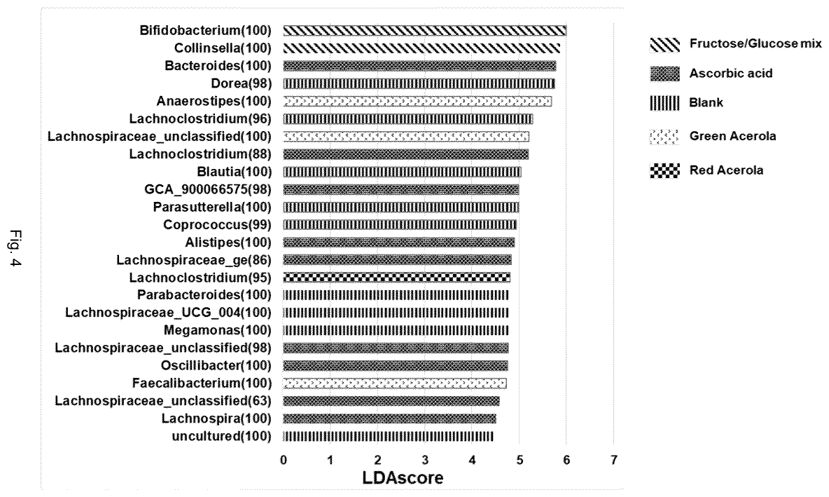

In another aspect, the present invention relates to a red acerola fruit extract for use in a method for promoting the growth of microorganisms of FIG. 4 in the gut microbiome.

In another aspect, the present invention relates to a green acerola fruit extract for use in a method for promoting the growth of microorganisms of FIG. 4 in the gut microbiome.

In a further aspect, the present invention relates to a method of increasing the population of genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome comprising directly delivering to the large intestine of red acerola fruit extract (hereinafter the third method of the invention).

In a certain embodiment, the person is experiencing or is at risk of experiencing high cholesterol levels.

In a further aspect, the present invention relates to a method of increasing the population of the genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome comprising directly delivering to the large intestine of green acerola fruit extract (hereinafter the fourth method of the invention).

In a further aspect, the present invention relates to a method of increasing the population of the bacteria from FIG. 4 in the gut microbiome comprising directly delivering to the large intestine of green acerola fruit extract.

In a further aspect, the present invention relates to a method of increasing the population of the bacteria from FIG. 4 in the gut microbiome comprising directly delivering to the large intestine of red acerola fruit extract.

In a certain embodiment, the person is experiencing or is at risk of experiencing at least one of the following conditions gastrointestinal infections, intestinal mucosal health problems, gut barrier integrity probelms, congenital chloride diarrhea, cholera, colorectal cancer, inflammatory bowel disease, colon heartburn, cancer, irritable bowel syndrome, histamine intolerance and lactose intolerance, gallstones, cholecystitis and cholangitis, rectal problems (anal fissure, haemorrhoids, proctitis and rectal prolapse).

In a certain embodiment or the third and fourth methods, the population of the genera and/or families was decreased prior to direct delivery of the acerola extract.

In a certain embodiment, a short chain fatty acid in the intestine is increased.

Use of red acerola fruit extract directly delivered to the large intestine to increase the population of genera, Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome (hereinafter the third use of the invention).

In a certain embodiment, the person is experiencing or is at risk of experiencing high cholesterol levels.

In a further aspect, the present invention relates to Use of red acerola fruit extract directly delivered to the large intestine to increase the population of the genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae in the gut microbiome comprising directly delivering to the large intestine of green acerola fruit extract (hereinafter the fourth use of the invention).

In a certain embodiment, the person is experiencing or is at risk of experiencing at least one of the following conditions gastrointestinal infections, intestinal mucosal health problems, gut barrier integrity probelms, congenital chloride diarrhea, cholera, colorectal cancer, inflammatory bowel disease, colon heartburn, cancer, irritable bowel syndrome, histamine intolerance and lactose intolerance, gallstones, cholecystitis and cholangitis, rectal problems (anal fissure, haemorrhoids, proctitis and rectal prolapse).

In a certain embodiment or the third and fourth methods, the population of the genera and/or families was decreased prior to direct delivery of the acerola extract.

In a certain embodiment, a short chain fatty acid in the intestine is increased.

The term “directly delivered” means that the acerola extract is formulated such that they are available to the lower intestine gut microflora. There are a variety of delayed-release or sustained-release forms which can accomplish this known in the art.

The term “treating” includes therapeutically treating or non-therapeutically treating.

The term “acerola” and “acerola fruit extract” has been defined or explained above, and this definition is applicable to the second third and fourth methods or uses of the invention. In a particular embodiment, the acerola fruit extract is an acerola fruit powder.

In a more particular embodiment, the acerola fruit extract is an acerola juice powder.

In another particular embodiment, the red acerola fruit extract contains vitamin C, antioxidant polyphenolic compounds, fiber, glucose, fructose and polyunsaturated fatty acids (PUFA).

The terms “vitamin C”, “polyphenolic compounds” and “PUFA” have been defined or explained above, and these definitions are applicable to the second method of the invention.

In a particular embodiment, the PUFA is a polyunsaturated fatty acid (PUFA), with 18 carbon atoms. In a more particular embodiment, the PUFA is selected from the group consisting of: alpha-linolenic acid (ALA), stearidonic acid (SDA), linoleic acid (LA), gamma-linolenic acid (GLA), oleic acid, a conjugated linoleic acid or a conjugated linolenic acid. In a more particular embodiment the conjugated linoleic acid is rumenic acid and the conjugated linolenic acid is selected of the group consisting of: α-calendic acid, β-Calendic acid, jacaric acid, α-eleostearic acid, β-eleostearic acid, catalpic acid, punicic acid, α-parinaric or β-parinaric acid.

In another particular embodiment, the PUFA is a long-chain polyunsaturated fatty acids (LC-PUFA) with 20 or more carbon atoms. In a more particular embodiment, the LC-PUFA is selected from the group consisting of: eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, cervonic acid), tetracosapentaenoic acid, eicosadienoic acid, arachidonic acid (AA) or adrenic acid (AdA).

In another particular embodiment, the red acerola fruit extract contains between 20% and 40% of vitamin C.

In another particular embodiment, the red acerola fruit extract contains between 4,5% and 15% of polyphenolic compounds. In yet another embodiment, the red acerola fruit extract contains between 110 and 120 mg of gallic acid equivalents per gram of extract.

In another particular embodiment, the red acerola fruit extract contains between 2% and 9% of total fiber,

In another particular embodiment, the red acerola fruit extract contains between 25% and 50% of the combined content of glucose and fructose

In another particular embodiment, the red acerola fruit extract contains between 1700 and 3500 ppm of PUFA. In a more particular embodiment, the red acerola fruit extract comprises a combination of polyunsaturated fatty acids (PUFA). In a preferred embodiment, the red acerola fruit extract comprises a combination of oleic acid (18:1), linoleic acid (18:2) and linolenic acid (18:3).

In another particular embodiment, the red acerola fruit extract may contain other components such as a pH regulator or a pH modifier, or an acidity regulator. In a particular embodiment, the acerola fruit extract contains magnesium hydroxide as an acidity regulator.

Bacteroides is a genus of Gram-negative, obligate anaerobic bacteria. Bacteroides species are non-endospore-forming bacilli, and may be either motile or non motile, depending on the species. Bacteroides species are normally mutualistic, making up the most substantial portion of the mammalian gastrointestinal microbiota, where they play a fundamental role in processing of complex molecules to simpler ones in the host intestine. The genus Bacteroides includes, but is not limited to, the species: B. acidifaciens, B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. intestinalis, B. luti, B. massiliensis, B. nordii, B. oleiciplenus, B. oris, B. ovatus, B. paurosaccharolyticus, B. plebeius, B. polypragmatus, B. propionicifaciens, B. putredinis, B. pyogenes, B. reticulotermitis, B. rodentium, B. salanitronis, B. salyersiae, B. sartorii, B. sediment, B. stercoris, B. suis, B. tectus, B. thetaiotaomicron, B. uniformis, B. vulgatus and B. xylanisolvens.

Anaerostipes is a Gram-positive and anaerobic bacterial genus from the family of Lachnospiraceae. The genus Anaerostipes includes, but is not limited to, the species: A. butyraticus, A. caccae, A. hadrus and A. rhamnosivorans.

Lachnoclostridium is a genus of Gram-positive, obligate anaerobic, spore-forming, motile bacteria. The Lachnoclostridium genus includes organisms from the Lachnospiraceae family and from several clostridial clusters such as Clostridium XIVa. The genus Lachnoclostridium includes, but is not limited to, the species: Lachnoclostridium phytofermentans, Lachnoclostridium bouchesdurhonense, Lachnoclostridium pacaense, Lachnoclostridium touaregense and Lachnoclostridium bouchesdurhonense.

Parasutterella is a genus of Gram-negative, circular/rod-shaped, obligate anaerobic, non-spore forming bacteria from the Pseudomonadota phylum, Betaproteobacteria class and the family Sutterellaceae. The Parasutterella genus includes, but is not limited to, the species: Parasutterella excrementihominis and Parasutterella secunda.

Bifidobacterium is a genus of gram-positive, nonmotile, often branched anaerobic bacteria. Bifidobacteria is one of the major genera of bacteria that make up the gastrointestinal tract microbiota in mammals. Some bifidobacteria are used as probiotics.

The genus Bifidobacterium includes, but is not limited to, the species: B. actinocoloniiforme, B. adolescentis, B. aemilianum, B. aerophilum, B. aesculapii, B. avesanii, B. bifidum, B. bohemicum, B. callimiconis, B. castoris, B. catenulatum, B. catulorum, B. cebidarum, B. choerinum, B. choladohabitans, B. choloepi, B. colobi, B. commune, B. criceti, B. crudilactis, B. cuniculi, B. dentium, B. eulemuris, B. faecale, B. globosum, B. goeldii, B. hapali, B. lemurum, B. leontopitheci, B. Iongum, B. magnum, B. margollesii, B. merycicum, B. olomucense, B. panos, B. parmae, B. primatium, B. pseudocatenulatum, B. pseudolongum, B. pullorum, B. ramosum, B. rousetti, B. ruminantium, B. saguini, B. saguinibicoloris, B. santillanense, B. scaligerum, B. scardovii, B. stellenboschense, B. subtile, B. thermacidophilum, B. thermophilum, B. tissieri and B. tsurumiense.

Faecalibacterium is a genus of gram positive bacteria. Its sole known species, Faecalibacterium prausnitzii is gram-positive, mesophilic, rod-shaped, anaerobic and is one of the most abundant and important commensal bacteria of the human gut microbiota.

Parabacteroides is a Gram-negative, anaerobic, non-spore-forming genus from the family Tannerellaceae. The genus includes, but is not limited to, the species: P. acidifaciens, P. bouchesdurhonensis, P. chartae, P. chinchilla, P. chongii, P. distasonis, P. faecis, P. goldsteinii, P. gordonii, P. johnsonii, P. massiliensis, P. merdae, P. pacaensis, P. provencensis and P. timonensis.

### UCG-002 Refers to the Genus Ruminococcaceae UCG-002.

Blautia is a genus of anaerobic bacteria with probiotic characteristics that occur widely in the feces and intestines of mammals. The genus includes, but is not limited to, the species: Blautia coccoides, Blautia hansenii, Blautia hydrogenotrophica, Blautia luti, Blautia product, Blautia schinkii, Blautia wexlerae, Blautia glucerasea, Blautia stercoris, Blautia faecis, Blautia obeum, Blautia caecimuris, Blautia massiliensis, Blautia phocaeensis, Blautia marasmi, Blautia provencensis, Blautia hominis, Blautia argi, Blautia brookingsii and Blautia faecicola.

CAG-56 refers to unknown bacteria that has not been named.

Alistipes is a genus in the phylum Bacteroidota and is a relatively new genus of bacteria isolated primarily from medical clinical samples. The genus Alistipes includes, but is not limited to, the species: Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, and Alistipes massiliensis. Alistipes communis and A. dispar.

The genus Phascolarctobacterium includes, but is not limited to, the species: Phascolarctobacterium faecium, Phascolarctobacterium succinatutens, Phascolarctobacterium wakonense.

Lachnospiraceae is a family of obligatory anaerobic, variably spore-forming bacteria in the order Eubacteriales that ferment diverse plant polysaccharides to short-chain fatty acids (butyrate, acetate) and alcohols (ethanol). These bacteria are among the most abundant taxa in the rumen and the human gut microbiota.

The red acerola fruit extract must be present in an effective amount to promote the growth of microorganisms of the genuses Bacteroides, Anaerostipes, Lachnoclostridium, Parasutterella, Bifidobacterium, Faecalibacterium, Parabacteroides, UCG-002, Blautia, CAG-56, Alistipes and Phascolarctobacterium or the family Lachnospiraceae in the gut microbiome. The term “effective amount” means any amount of the red acerola fruit extract which, when administered to a subject, is sufficient to produce the desired effect. The effective amount may vary depending on, for example, the age, body weight, general health, sex and diet of the subject, as well as the mode and timing of administration.

For example, an effective amount may be from about 0.5 g to 10 g of red acerola extract, such as from about 4 to 8 g, such as 6 g of acerola extract.

In another aspect, the present invention relates to a green acerola fruit extract for use in a method (hereinafter the third method of the invention) for promoting the growth of microorganisms of the genuses Bacteroides, Anaerostipes, Lachnoclostridium, Parasutterella, Bifidobacterium, Faecalibacterium, Parabacteroides, Dorea, UCG-002, Blautia, and Alistipes, and of the family Lachnospiraceae in the gut microbiome.

The term “acerola” and “acerola fruit extract” has been defined or explained above, and this definition is applicable to the third method of the invention. In a particular embodiment, the acerola fruit extract is an acerola fruit powder. In a more particular embodiment, the acerola fruit extract is an acerola juice powder.

In another particular embodiment, the green acerola fruit extract contains vitamin C, antioxidant polyphenolic compounds, fiber, glucose, fructose and polyunsaturated fatty acids (PUFA). The terms “vitamin C”, “polyphenolic compounds” and “PUFA” have been defined or explained above, and these definitions are applicable to the third method of the invention.

In a particular embodiment, the green acerola fruit extract contains between 30% and 60% of vitamin C.

In another particular embodiment, the green acerola fruit extract contains between 6% and 30% of polyphenolic compounds. In yet another embodiment, the green contains between 240 and 250 mg of gallic acid equivalents per gram of extract.

In another particular embodiment, the green acerola fruit extract contains between 0.5% and 4% of total fiber.

In another particular embodiment, the green acerola fruit extract contains between 5% and 15% of the combined content of glucose and fructose.

In another particular embodiment, the green acerola fruit extract may contain other components such as a pH regulator or a pH modifier, or an acidity regulator. The term “pH regulator” or “acidity regulator” has been defined or explained above, and this definition is applicable to the third method of the invention.

As mentioned above, the use of a red or a green acerola fruit extract results in a different microbial composition. Some of the genuses that grow using the green acerola fruit extract are the same genuses which grow using the red acerola fruit extract, i.e., Bacteroides, Anaerostipes, Lachnoclostridium, Lachnospiracea, Bifidobacterium, Faecalibacterium, Parabacteroides, UCG-002, Blautia, and Alistipes. The green acerola fruit extract promotes the growth of the genuses Parasutterella and Dorea.

Parasutterella is a genus of Gram-negative, circular/rod-shaped, obligate anaerobic, non-spore forming bacteria from the Pseudomonadota phylum, Betaproteobacteria class and the family Sutterellaceae. The genus Parasutterella includes, but is not limited, to the species: Parasutterella excrementihominis and Parasutterella secunda.

Dorea is a Gram-positive and non-spore-forming bacterial genus from the family Lachnospiraceae. The genus Dorea includes, but is not limited to, the species: D. acetigenes, D. ammoniilytica, D. formicigenerans, D. hominis and D. Iongicatena.

The green acerola fruit extract must be present in an effective amount to promote the growth of microorganisms of the genuses Bacteroides, Anaerostipes, Lachnoclostridium, Parasutterella, Bifidobacterium, Faecalibacterium, Parabacteroides, Dorea, UCG-002, Blautia, and Alistipes, and of the family Lachnospiraceae in the gut microbiome. The term “effective amount” means any amount of the green acerola fruit extract which, when administered to a subject, is sufficient to produce the desired effect. The effective amount may vary depending on, for example, the age, body weight, general health, sex and diet of the subject, as well as the mode and timing of administration. For example, an effective amount may be from about 0.5 g to 10 g of green acerola extract, such as from about 4 to 8 g, such as 6 g of acerola extract.

The authors of the present invention have discovered that the use of a green acerola extract or a red acerola extract promotes the growth of some genera of microorganisms.

Thus, in a particular embodiment, the method which comprises the use of a green acerola fruit extract or a red acerola fruit extract promotes the growth of microorganisms belong to the Anaerostipes, Bifidobacterium, and/or Faecalibacterium genera.

The acerola fruit extract must be present in an effective amount to promote the growth of microorganisms belong to the Anaerostipes, Bifidobacterium, and/or Faecalibacterium genera. The term “effective amount” means any amount of the acerola fruit extract which, when administered to a subject, is sufficient to produce the desired effect. The effective amount may vary depending on, for example, the age, body weight, general health, sex and diet of the subject, as well as the mode and timing of administration. For example, an effective amount may be from about 0.5 g to 10 g of acerola extract, such as from about 4 to 8 g, such as 6 g of acerola extract.

The Anaerostipes, Bifidobacterium, and/or Faecalibacterium genera have been shown to have various health benefits.

Faecalibacterium prausnitzii is one of the most abundant bacterial species of the colon and changes to population abundance have been observed in many diseases. It has digestive health benefits, such as maintains the intestinal health, controls inflammation through inhibition of inflammatory cytokines, has positive effects on insulin resistance and protects mucous membranes.

Bifidobacteria have many digestive health benefits, such as the prevention of colon cancer, mitigation of diarrhea, reduction in symptoms of inflammatory bowel disease, and increasing regularity. In addition, Bifidobacteria can prevent gastrointestinal infections by competitive exclusion of pathogens based on common binding sites of epithelial cells. Bifidobacteria are capable of digesting fiber, leading to the generation of SCFA and other metabolites such as B vitamins and essential fatty acids that may support overall health of the host.

Anaerostipes have digestive health benefits such as, helps to the treatment of food allergies in humans and the protection against colon cancer. The term “food allergies” refers to an immune system reaction that occurs soon after eating a certain food. Food allergic reactions vary in severity from mild symptoms involving hives and lip swelling to severe, life-threatening symptoms, often called anaphylaxis that may involve fatal respiratory problems and shock.

Thus, in a particular embodiment, the use of the red acerola fruit extract or the green acerola fruit extract that promotes the growth of the Anaerostipes, Bifidobacterium, and/or Faecalibacterium genera results in the treatment or prevention of digestive diseases, increases the levels of SCFAs and other metabolites such as B vitamins or essential fatty acids, or helps to the treatment of food allergies.

In another particular embodiment, the use of the red acerola fruit extract or the green acerola fruit extract that promotes the growth of the Anaerostipes, Bifidobacterium, and/or Faecalibacterium genera results in the prevention of gastrointestinal infections.

The term “prevention” has been defined above.

The term “gastrointestinal infections” refers to viral, bacterial or parasitic infections that cause gastroenteritis or an inflammation of the gastrointestinal tract involving both the stomach and the small intestine.

In a particular embodiment, the gastrointestinal infection is caused by a bacteria. In a more particular embodiment, the bacterial gastrointestinal infection is caused by Salmonella, Escherichia, Clostridium, Listeria or Staphylococcus.

In another particular embodiment, the gastrointestinal infection is caused by a virus. In a more particular embodiment, the viral gastrointestinal infection is caused by a Norovirus or Rotavirus. Noroviruses are the most common cause of foodborne illness worldwide. Rotavirus is the leading cause of viral gastroenteritis in children worldwide.

In another particular embodiment, the gastrointestinal infection is caused by intestinal helminths, worms, or protozoan parasites. In a more particular embodiment, the parasitic gastrointestinal infection is giardiasis, caused by Giardia duodenalis (G. lamblia, G. intestinalis) or cryptosporidiosis, caused by Cryptosporidium.

The acerola fruit extract must be present in an effective amount to prevent gastrointestinal infections. The term “effective amount” means any amount of the acerola fruit extract which, when administered to a subject, is sufficient to produce the desired effect. The effective amount may vary depending on, for example, the age, body weight, general health, sex and diet of the subject, as well as the mode and timing of administration.

The acerola fruit extract can be provided to a subject in a variety of forms. For example, the acerola fruit extract can be provided as a nutraceutical composition, as a cosmetic composition, as a food supplement, as a cosmeceutical composition or as a dietary supplement.

Thus, in another aspect, the present invention relates to a cosmetic, nutraceutical or cosmeceutical composition (hereinafter the compositions of the invention), a food supplement or dietary supplement (hereinafter the supplements of the invention) comprising an acerola fruit extract.

The term “acerola fruit extract” has been defined or explained above, and this definition is applicable to the compositions of the invention and to the supplements of the invention. In a particular embodiment, the acerola fruit extract is an acerola fruit powder. In a more particular embodiment, the acerola fruit extract is an acerola juice powder.

In another particular embodiment, the acerola fruit extract is red acerola fruit extract or green acerola fruit extract.

In another particular embodiment, the acerola fruit extract contains vitamin C, antioxidant polyphenolic compounds, fiber, glucose, fructose and polyunsaturated fatty acids (PUFA). The terms “vitamin C”, “polyphenolic compounds” and “PUFA” have been defined or explained above, and these definitions are applicable to the compositions of the invention and to the supplements of the invention.

In a particular embodiment, the PUFA is a polyunsaturated fatty acids (PUFA), with 18 carbon atoms. In a more particular embodiment, the PUFA is selected from the group consisting of: alpha-linolenic acid (ALA), stearidonic acid (SDA), linoleic acid (LA), gamma-linolenic acid (GLA), oleic acid, a conjugated linoleic acid or a conjugated linolenic acid. In a more particular embodiment the conjugated linoleic acid is rumenic acid and the conjugated linolenic acid is selected of the group consisting of: α-calendic acid, β-Calendic acid, jacaric acid, α-eleostearic acid, β-eleostearic acid, catalpic acid, punicic acid, α-parinaric or β-parinaric acid.

In another particular embodiment, the PUFA is a long-chain polyunsaturated fatty acids (LC-PUFA) with 20 or more carbon atoms. In a more particular embodiment, the LC-PUFA is selected from the group consisting of: eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, cervonic acid), tetracosapentaenoic acid, eicosadienoic acid, arachidonic acid (AA) or adrenic acid (AdA). In another particular embodiment, the acerola fruit extract contains at least 20% vitamin C. In a more particular embodiment, the acerola fruit extract contains between 20% and 60% of vitamin C.

In another particular embodiment, the acerola fruit extract contains at least 4,5% of antioxidant polyphenolic compounds. In a more particular embodiment, the acerola fruit extract contains between 4,5% and 30% of polyphenolic compounds. In yet another embodiment, the acerola fruit extract contains between 100 and 300 mg of gallic acid equivalents per gram of extract.

In another particular embodiment, the acerola fruit extract contains at least 0.5% of total fiber. In a more particular embodiment, the acerola fruit extract contains between 0.5% and 10% of total fiber.

In another particular embodiment, the acerola fruit extract contains at least 5% of the combined content of glucose and fructose. In a more particular embodiment, the acerola fruit extract contains between 5% and 50% of the combined content of glucose and fructose.

In another particular embodiment, the acerola fruit extract contains at least 1000 ppm of PUFA. In a more particular embodiment, the acerola fruit extract contains between 1000 ppm and 4000 ppm of PUFA.

The quantity of vitamin C, antioxidant polyphenolic compounds, fiber, glucose, fructose and polyunsaturated fatty acids (PUFA) may differs between the red acerola fruit extract and green acerola fruit extract.

Thus, in a particular embodiment, the green acerola fruit extract contains between 30% and 60% of vitamin C.

In another particular embodiment, the green acerola fruit extract contains between 6% and 30% of polyphenolic compounds. In yet another embodiment, the green acerola fruit extract contains between 240 and 250 mg of gallic acid equivalents per gram of extract.

In another particular embodiment, the green acerola fruit extract contains between 0.5% and 4% of total fiber.

In another particular embodiment, the green acerola fruit extract contains between 5% and 15% of the combined content of glucose and fructose.

In another particular embodiment, the red acerola fruit extract contains between 20% and 40% of vitamin C.

In another particular embodiment, the red acerola fruit extract contains between 4,5% and 15% of polyphenolic compounds. In yet another embodiment, the red acerola fruit extract contains between 110 and 120 mg of gallic acid equivalents per gram of extract.

In yet another embodiment, the acerola fruit extract is a green acerola fruit extract that contains between 240 and 250 of gallic acid equivalents per gram of extract.

In another particular embodiment, the red acerola fruit extract contains between 2% and 9% of total fiber,

In another particular embodiment, the red acerola fruit extract contains between 25% and 50% of the combined content of glucose and fructose

In another particular embodiment, the red acerola fruit extract contains between 1700 and 3500 ppm of PUFA. In a more particular embodiment, the red acerola fruit extract comprises a combination of polyunsaturated fatty acids (PUFA). In a preferred embodiment, the red acerola fruit extract comprises a combination of oleic acid (18:1), linoleic acid (18:2) and linolenic acid (18:3).

In another particular embodiment, the acerola fruit extract may contain other components such as a pH regulator or a pH modifier, or an acidity regulator. In a more particular embodiment, the acerola fruit extract contains magnesium hydroxide as an acidity regulator.

The composition, broadly defined, is a set of components consisting of at least the acerola fruit extract. The composition may comprise other components such as, for example, inert substances or bioactive components.

In a particular embodiment, the compositions may further comprise at least one bioactive component (active substance, active principle or therapeutic agent), such as for example other food components, plant or fungal products and/or pharmaceuticals.

The term “bioactive component” refers to a compound with biological activity within the scope of the patent that may enhance or complement the activity of the acerola fruit extract, including food ingredients or components, probiotics, plants, fungi, extracts or components of plants, fungi and pharmaceuticals.

The composition of the invention may be formulated (i) for dietary administration, i.e. on its own or as part of the food consumed in the diet of the subject, (ii) for cosmetic administration, i.e. on its own or as part of cosmetic products or (iii) for cosmeceutical administration, i.e. on its own or as part of cosmetic products administered to the subject by any means of administration.

The term “subject” refers to any animal of any species. Preferably the subject is a mammal. More preferably the subject is a human.

In a particular aspect, the present invention relates to a cosmetic composition which comprises an acerola fruit extract. The term “cosmetic composition” as used herein, refers to any substance or mixture intended to be placed in contact with the superficial parts of the human body (epidermis, hair and capillary system, nails, lips and external genital organs) or with the teeth and oral mucous membranes, for the sole or principal purpose of cleaning them, perfuming them, changing their appearance, protecting them, keeping them in good condition or correcting body odours.

In a particular embodiment, the present invention relates to the use of the acerola fruit extract for the production of cosmetic products.

In another particular aspect, the present invention relates to a nutraceutical composition which comprises an acerola fruit extract. The term “nutraceutical composition” refers to foods or naturally occurring food supplements intended for human ingestion, that have medical or health benefits, including the prevention and treatment of a disease. The nutraceutical composition may be a food or incorporated into a food or foodstuff intended for human and/or animal consumption. Thus, in a particular embodiment, the nutraceutical composition is a food or a nutritional supplement.

In some particular embodiments, the nutraceutical composition of the invention is a food for specific nutritional purposes. In some specific embodiments, the nutraceutical composition of the invention is a food for special medical purposes. Foods for special medical purposes are those foods that have been specially prepared or formulated for the dietary management of patients under medical supervision. These foods are intended to meet all or part of the dietary needs of patients.

Examples of foods that may comprise the nutraceutical composition include, but are not limited to, animal feed, dairy products, vegetable products, meat products, snacks, chocolates, beverages, baby food, cereals, fried foods, industrial bakery products and biscuits. Examples of dairy products include, but are not limited to, fermented milk products (e.g., but not limited to, yoghurt or cheese) or non-fermented milk (e.g., but not limited to, ice cream, butter, margarine or whey). The plant product is, for example, but not limited to, a cereal in any form of presentation, fermented (e.g., soy yoghurt, oat yoghurt, etc.) or unfermented. Beverages include, but are not limited to, any fruit juice or non-fermented milk.

In another particular aspect, the present invention relates to a cosmeceutical composition, which comprises an acerola fruit extract. The term “cosmeceutical composition” as used herein refers to a cosmetic composition that have medicinal or drug-like benefits. This composition has both cosmetic and pharmaceutical properties.

The term “drugs” refer to products that cure, treat, mitigate or prevent disease or that affect the structure or function of the human body. The term “pharmaceutical properties” refers to the improvement of the physical or physiological or psychological well-being of a subject which implies an improvement in the general state of health or a reduction in the risk of disease.

The compositions can be administered orally, topically, parenterally, rectally, ophthalmically, otic and/or nasally, among others.

The presentation of the compositions will be adapted to the type of administration used. The compositions may be formulated in solid, semi-solid or liquid preparations, such as tablets, capsules, powders, granules, solutions, suppositories, gels, microspheres and/or any other form known in the food, medical device, cosmetic and/or pharmaceutical art.

In a particular embodiment, the compositions are formulated for administration in solid or liquid form.

In another particular embodiment, the solid formulation is selected from the group consisting of tablets, lozenges, candies, chewable tablets, chewing gums, capsules, sachets, powders, granules, coated particles or coated tablets, tablets, pills, gastro-resistant tablets and capsules, and dispersible strips and films.

In another particular embodiment, the liquid formulation is selected from the group consisting of oral solutions, suspensions, emulsions and syrups.

Furthermore, various systems are known which can be used for sustained release administration of the compositions, including, without limitation, encapsulation in liposomes, microbubbles, microparticles or microcapsules and the like. Suitable sustained release forms, as well as materials and methods for their preparation, are well known in the prior art. Thus, the orally administrable form of the compositions is in a sustained release form further comprising at least one coating or matrix. The sustained release coating or matrix includes, without limitation, semi-synthetic or synthetic, water insoluble or modified natural polymers, waxes, fats, fats, fatty alcohols, fatty acids, natural, semi-synthetic or synthetic plasticisers or a combination of two or more thereof.

Enteric coatings can be applied by conventional processes known to the person skilled in the art.

In addition to what is described above, the present invention also covers the possibility that the compositions may be administered to a subject together with other components or compounds, even if these do not form part of the composition.

In another particular aspect, the present invention relates to a food supplement which comprises an acerola fruit extract. The term “food supplement” refers to products or preparations intended to supplement the normal diet of a subject and consisting of concentrated nutrient sources or other substances with a nutritional or physiological effect.

In another particular aspect, the present invention relates to a dietary supplement which comprises an acerola fruit extract. The term “dietary supplement” refers to a manufactured product intended to supplement the dietary needs of patients.

The food supplement or the dietary supplement may be in single or combined form and marketed in dosage form, i.e. in capsules, pills, tablets and other similar forms, powder sachets, liquid ampoules and drop dispensing bottles and other similar forms of liquids and powders designed to be taken in a single quantity. There is a wide range of nutrients and other elements that may be present in food supplements, including vitamins, minerals, amino acids, essential fatty acids, fibre, enzymes, plants, plant extracts, mushrooms and fungal extracts. Since their function is to supplement the nutrient supply of the diet, they should not be used as a substitute for a balanced diet and their intake should not exceed the daily dose expressly recommended by a doctor or nutritionist.

### Examples

Having now generally described the invention, the same will be more readily understood through reference to the following examples, which are provided by way of illustration and are not intended to be limiting of the present invention.

The authors of the present invention have investigated the biological effects of an acerola fruit extract and impact of these products on the human gut microbiome.

### Materials and Methods

### Pre-Digestion

The test products used in these experiments included a red acerola fruit powder containing 20% vitamin C and a green acerola fruit powder containing 34% vitamin C provided by Naturex-Givaudan. Commercial synthetic ascorbic acid and a 55:45 mixture of fructose and glucose served as relevant control groups. The test products contained a fraction of digestible compounds (sugar, for e.g.) that, in vivo, are absorbed in the small intestine following conversion to small molecules by digestive enzymes. Hence, a pre-digestion step was employed to simulate the digestion process that takes place in the upper gastrointestinal tract. Stock solutions of test products and controls were prepared and exposed to conditions simulating oral, gastric, and small intestinal passage. Typically, a dialysis procedure is employed to simulate absorption of small molecules in the small intestine, but this step was omitted for these test products, as the dialysis procedure would lead to losses of compounds in acerola (polyphenols) that would reach the colon in vivo and exert an effect on the microbiome.

The acerola fruit powders contain approximately 50% sugar, predominantly as fructose (55%) and glucose (45%). As such, the fructose/glucose mixture was included in the experiment to control for the sugar found in acerola that would enter the colon simulation model, which would not occur in vivo.

### Short Term Colonic Simulation

A 48 hours colonic fermentation was performed in the presence of test compounds and relevant controls to determine the effect of the test products on the gut microbiome. Products and controls were tested in triplicate against the fecal matter from a single human donor. A reference condition not containing test products (negative control) was also included to assess baseline fermentation parameters. Dosing scheme for the study is provided in the following table:

TABLE 1
Overview of the in vitro product dose

| Product | Dose |
|---|---|
| Red Acerola | 3.5 g/L |
| Green Acerola | 3.5 g/L |
| Sugar control (55% fructose:45% glucose) | 1.75 g/L |
| Ascorbic acid | 1.67 g/L |

The sugar control used try to represent the ratio of fructose and glucose, and the dose mimics the dose of sugar found in the acerola products that would be contained in the fermentation system. The ascorbic acid dose represents an oral dose of 2 mg in a human (the upper limit for this vitamin) and 50% absorption in the small intestine, which would be expected when a high-dose of vitamin C is consumed in vivo.

At the start of the colonic fermentation experiment, the 4 pre-digested products and controls were added to individual reactors in triplicate. A carbohydrate depleted nutritional medium (containing basal nutrients of the colon including peptone, yeast extract and L-cysteine) was added to each reactor along with 10% (volume ratio) of a freshly prepared 75 g/L fecal inoculum from the selected healthy donor. The resulting final volume in the reactors was 70 mL. Simulation of the mucus layer was performed by inserting five mucus-coated carriers into each reactor. Reactors were then closed with a rubber septum, made anaerobic through flushing with nitrogen and were incubated for 48 hours under continuous shaking (90 rpm) in a temperature controlled shaking incubator.

### Endpoint Analysis

Sampling was performed periodically with a syringe through a rubber septum. Assessments of change in pH, gas production, short-chain fatty acid (SCFA) production, lactate production, and branched-chain fatty acid (BCFA) production were made at the start of the incubation (0 hours) and after 6, 24, and 48 hours. Changes in ammonium production were assessed by sampling at the start of incubation and after 48 hours. Analysis of microbial community structure was performed at the start of the incubation and after 48 hours.

### Overall Fermentative Activity: pH and Gas Production

pH was measured in each reactor at each time point as a single repetition. Gas production was measured in the headspace of the fermentation vessels as a single repetition.

### Microbial Metabolite Production

Quantitative analysis of the SCFA was done by means of capillary gas chromatography, coupled with a flame ionization detector (FID). The isolation of SCFA was performed by liquid-liquid extraction. Each measurement was done in single repetition. Determination of lactate concentrations was performed using the Enzytec™ kit (R-Biopharm). Each measurement was done in single repetition.

### Changes in Community Composition by 16S-Targeted Illumina Sequencing

The methodology applied involves primers that span 2 hypervariable regions (V3-V4) of the 16S rRNA gene, i.e. primers 341F-785R described in Bekliz et al (Bekliz, M., Pramateftaki, P., Battin, T. J. et al. Viral diversity is linked to bacterial community composition in alpine stream biofilms. ISME COMMUN. 2, 27 (2022). https://doi.org/10.1038/s43705-022-00112-9). Using a pair-end sequencing approach, sequencing of 2×250 bp resulted in 424 bp amplicons. Such fragments are taxonomically more informative than smaller fragments. Read assembly and cleanup was largely derived from the MiSeq SOP described by the Schloss lab. Briefly, mothur (v.1.44.3) was used to assemble reads into contigs, perform alignment-based quality filtering (alignment to the mothur-reconstructed SILVA SEED alignment, v138), remove chimeras (vsearch v2.13.3), assign taxonomy using a naïve Bayesian classifier and SILVA NR v138_1 and cluster contigs into OTUs at 97% sequence similarity. All sequences that were classified as Eukaryota, Archaea, Chloroplasts and Mitochondria were removed. Also, if sequences could not be classified at all (even at (super)Kingdom level) they were removed. The most abundant sequence within an OTU was picked as the representative. Reads with maximum abundances of only 5 across samples were removed, as they were supposedly artefacts or bacteria that were not having any biological impact.

### Statistics

### Metabolites

To assess whether treatment effects in terms of the investigated endpoints were significantly different between treatment and the reference condition (blank or product control), unpaired two-sided T-tests were performed. An effect was considered significant if the obtained β-value was below 0.05.

### Community Composition

For analysis of community composition, a different approach was followed. LEfSe was performed to identify the OTUs most likely to explain differences between the treatments. The extent of the difference in abundance is expressed by the LDA score. LEfSe couples statistical significance with biological consistency and effect size estimation and thus provides in-depth insight in the magnitude of an observed phenomenon. All features shown meet p 50.05 for Kruskal-Wallis and Wilcoxon tests and have an LDA score 2.0.

### Results

### pH and Gas Production

Major pH shifts occurred within the 0-6 hours timeframe for both acerola products, suggesting efficient substrate fermentation. The magnitude of the shift for both acerola products was not greater than that of the sugar control which contained much higher sugar concentration than the acerola extracts, it is likely that the shift is largely due to the presence of sugar in the products. Ascorbic acid, however, failed to produce a significant shift in pH relative to the other products.

Each product of the acerola group (including the sugar control) stimulated gas production, but no major differences between products were observed. This suggests that gas production was mostly driven by the sugar background. Also, gas production between 0-6 h was mild and not expected to lead to discomfort.

### Microbial Metabolites

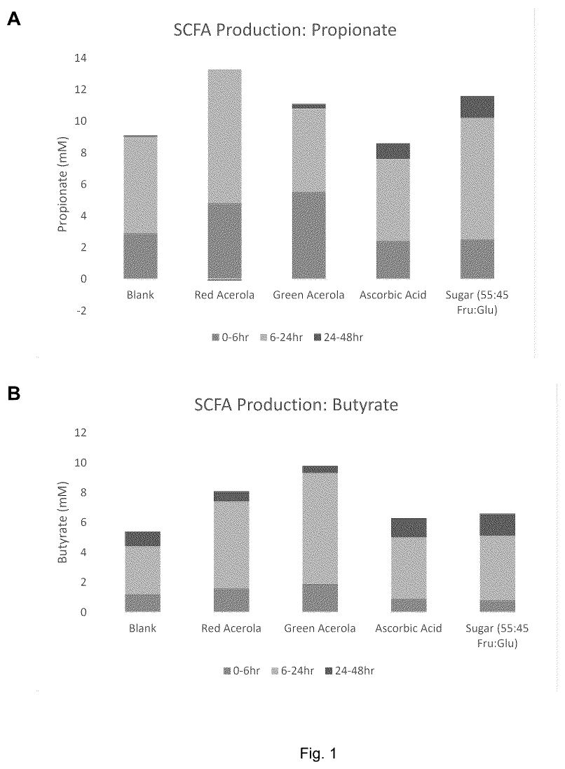

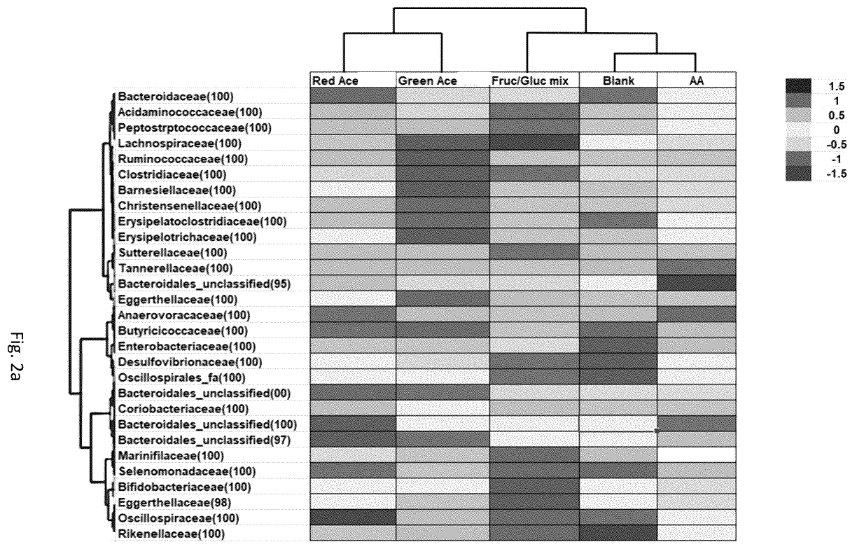

Each product, except ascorbic acid, stimulated propionate production (FIG. 1). The strongest propionogenic effect was attributed to red acerola. Green acerola yielded similar propionate levels as the sugar control. These data suggest that something in the red acerola matrix (polyphenols, possibly) stimulates propionate production, and this effect extends beyond that of a high-dose of synthetic ascorbic acid alone.

Each product stimulated butyrate production. The strongest butyrogenic effects were attributed to green acerola, followed by red acerola. Each of these products yielded significantly more butyrate than the sugar control. These data suggest that something in the acerola matrix stimulates butyrate production, and this effect extends beyond that of a high-dose of synthetic ascorbic acid alone.

### Microbial Community Structure

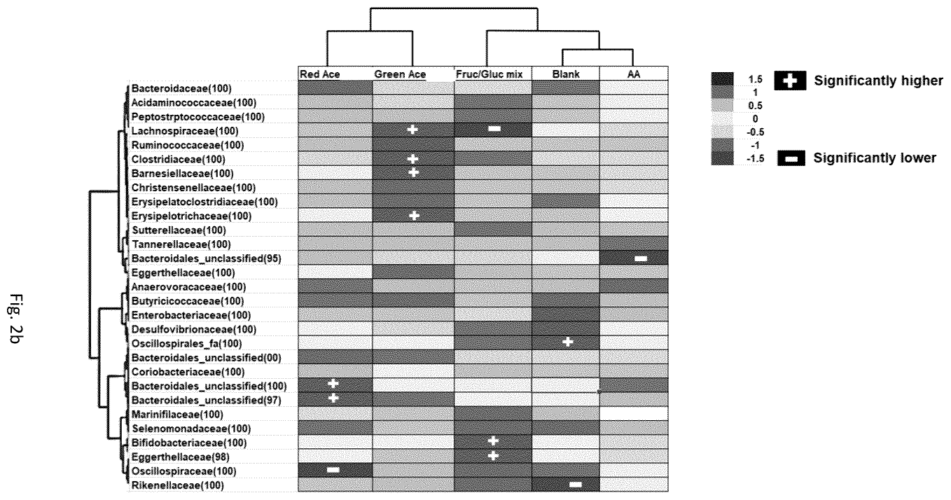

A heatmap (FIG. 2) displaying the abundances of different bacterial families after the treatments was constructed. Based on the obtained output, samples were clustered to reveal similarities in community composition between treatments. Overall, a broad bacterial spectrum was enriched by the various treatments (mostly by the acerola products). The data show a different community composition between green/red acerola and the product control (fructose/glucose mix), clearly indicating that the non-absorbable product fraction triggered community shifts. The graph reveals similar patterns of enrichment for green and red acerola (with some differences), which are distinct from ascorbic acid. These results further support the notion that the acerola matrix contains compounds that stimulate the microbiota differently than synthetic ascorbic acid alone.

LEfSe detected many significant differences in community composition between red acerola and its sugar background, suggesting that the non-absorbable product fraction triggers community shifts. Amongst those, the strongest enrichments involved OTUs of the genera Bacteroides, Anaerostipes, Lachnoclostridium, Lachnospiraceae, Parasutterella, Bifidobacterium, Faecalibacterium, Parabacteroides, UCG-002, Blautia, CAG-56, Alistipes and Phascolarctobacterium. LEfSe detected many significant differences in community composition between green acerola and its sugar background, suggesting that the non-absorbable product fraction triggers community shifts. Amongst those, the strongest enrichments involved OTUs of the genera Bacteroides, Anaerostipes, Lachnoclostridium, Lachnospiraceae, Parasutterella, Bifidobacterium, Faecalibacterium, Parabacteroides, Dorea, UCG-002, Blautia, and Alistipes. The strongest enrichments induced by ascorbic acid involved OTUs of the genera/families Bacteroides, Lachnoclostridium, GCA-900066575, Anaerostipes, Agathobacter, Oscillibacter, Alistipes, Lachnospiraceae, Flavonifractor, Coprococcus and Blautia. Taken together, these results suggest that the acerola matrix itself may promote the growth of Anaerostipes, Bifidobacteria, and Faecalibacterium genera, all of which positively influence gut health. Promotion of these genera is not attributed to vitamin C.

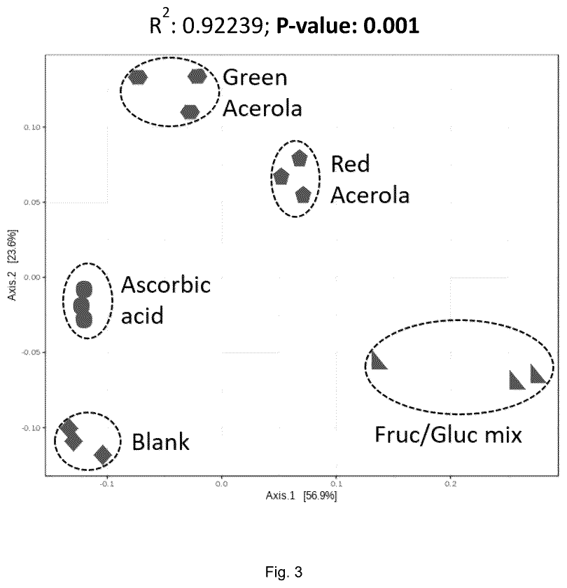

Beta diversity analysis reveal clear divergence between green/red acerola samples and controls (FIG. 3).

Linear discrimination analysis effect size analysis for microbiome biomarker identification revealed upregulation of beneficial genera in acerola groups (FIG. 4)

1. A method for promoting a propionogenic or butyrogenic effect in the gut of a subject or for increasing the production of SCFAs by the gut microbiota in a subject, the method comprising administering an acerola fruit extract to said subject.

2. The method according to claim 1, wherein the acerola fruit extract is red acerola fruit extract and wherein the effect is propionogenic.

3. The method according to claim 2, wherein the method provides a reduction of the cholesterol levels, a reduction of the fatty acid levels, or an increase in satiety in said subject.

4. The method according to claim 1, wherein the acerola fruit extract is green acerola fruit extract and wherein the effect is butyrogenic.

5. The method according to claim 4, wherein the method provides a health benefit selected from at least one of: restoring intestinal mucosal health, increases gut barrier integrity, results in the symptomatic treatment of congenital chloride diarrhea, results in the symptomatic treatment of cholera, results in the treatment or prevention of colorectal cancer or results in the prevention or treatment of an inflammatory bowel disease of the colon in said subject.

6. The method according to claim 5, wherein the intestinal mucosal health is altered due to acute gastroenteritis.

7. The method according to claim 5, wherein the inflammatory bowel disease is ulcerative colitis or Crohn's disease.

8. A non-therapeutic method for increasing gastrointestinal regularity or for increasing the SCFA content, B vitamins and/or essential fatty acids in a subject, the method comprising administering to the subject an acerola fruit extract.

9. The non-therapeutic method according to claim 8, wherein the acerola fruit extract is red acerola fruit extract or green acerola fruit extract.

10. A method for promoting the growth of microorganisms of the genuses Bacteroides, Anaerostipes, Lachnoclostridium, Parasutterella, Bifidobacterium, Faecalibacterium, Parabacteroides, UCG-002, Blautia, Alistipes and Phascolarctobacterium, of the family Lachnospiraceae and/or of the CAG-56 bacteria in the gut microbiome, the method comprising administering a red acerola fruit extract to a subject;
OR
for promoting the growth of microorganisms of the genuses Bacteroides, Anaerostipes, Lachnoclostridium, Parasutterella, Bifidobacterium, Faecalibacterium, Parabacteroides, Dorea, Ruminococcaceae UCG-002, Blautia, and Alistipes and of the family Lachnospiraceae, the method comprising administering a green acerola fruit extract to said subject.

11. (canceled)

12. The method of claim 10, wherein the microorganisms belong to the Anaerostipes, Bifidobacterium, and/or Faecalibacterium genera.

13. The method of claim 12, wherein the method results in the prevention of gastrointestinal infections.

14. A method of increasing the population of genuses Bacteroides, Anaerostipes, Lachnoclostridium, Parasutterella, Bifpdobacterium, Faecalibacterium, Parabacteroides, Dorea, Ruminococcaceae UCG-002, Blautia, and Alistipes and of the family Lachnospiraceae in the gut microbiome comprising directly delivering to the large intestine of a subject of red acerola fruit extract and/or green acerola fruit extract.

15. (canceled)

16. The method according to claim 14, wherein the population of the genera and/or families was decreased prior to direct delivery of the acerola extract.

17. The method according to claim 14, wherein the subject is experiencing or is at risk of experiencing high cholesterol levels.

18. The method according to claim 14, wherein the subject is experiencing or is at risk of experiencing at least one of the following conditions: gastrointestinal infections, intestinal mucosal health problems, gut barrier integrity problems, congenital chloride diarrhea, cholera, colorectal cancer, inflammatory bowel disease, colon heartburn, cancer, irritable bowel syndrome, histamine intolerance and lactose intolerance, gallstones, cholecystitis and cholangitis, rectal problems elected from anal fissure, hemorrhoids, proctitis and rectal prolapse.

19. The method according to claim 14, wherein a short chain fatty acid in the intestine is increased.

20. The method of claim 1, wherein the extract contains at least 20% vitamin C, wherein the extract contains at least 4.5% of antioxidant polyphenolic compounds, wherein the extract contains at least 0.5% of total fiber, wherein the combined content of glucose and fructose in the extract is of at least 5% and/or wherein the PUFA content is of at least 1000 ppm.
